(19) United States
(12) Patent Application Publication
(10) Pub. No.: US 2024/0181297 A1
(43) Pub. Date: Jun. 6, 2024

(54) LIGHT TOUCH KEEPING APPARATUS, LIGHT TOUCH KEEPING METHOD AND PROGRAM

(71) Applicant: NIPPON TELEGRAPH AND TELEPHONE CORPORATION, Tokyo (JP)

(72) Inventors: Masato SHINDO, Tokyo (JP); Takashi ISEZAKI, Tokyo (JP); Ryosuke AOKI, Tokyo (JP)

(21) Appl. No.: 18/554,316

(22) PCT Filed: Apr. 15, 2021
(86) PCT No.: PCT/JP2021/015634

§ 371 Date: Oct. 6, 2023

(51) Int. Cl. A63B 26/00

(52) U.S. Cl. CPC A63B 26/003; A63B 2220/51; A63B 2220/56; A63B 2220/58; A63B 2230/625

(57) ABSTRACT

A light touch maintaining device includes: a contact load evaluation unit that determines whether a subject person is in a light touch state, on the basis of a contact load between the subject person and a support; and an exercise intervention control unit that controls exercise intervention on the subject person, to maintain a light touch state when the subject person is not in a light touch state.

### TECHNICAL FIELD

The present invention relates to a technology for maintaining a light touch state.

### BACKGROUND ART

The effect of reducing gravity center sway when a person lightly touches a support such as a wall or a bar with a force (1 N or smaller) that does not dynamically support a fingertip is called a light touch effect. When a light touch effect is applied in rehabilitation training or the like for maintaining an upright posture, posture stability can be improved.

In a conventional technology, various kinds of sensory feedback are used as methods for maintaining a contact load of 1 N or smaller between a fingertip and a support in a light touch state. For example, Non Patent Literature 1 discloses a technique for sending feedback, by sound, about a fact that the contact load between a fingertip and a force plate is larger than 1 N.

### CITATION LIST

### Non Patent Literature

Non Patent Literature 1: Marco Baccini, Lucio A. Rinaldi, Gianluca Federighi, Luca Vannucchi, Matteo Paci, Giulio Masotti, “Effectiveness of fingertip light contact in reducing postural sway in older people”, Age and Ageing, Volume 36, Issue 1, January 2007, Pages 30-35.

### SUMMARY OF INVENTION

### Technical Problem

In a case where the subject person performs a highly difficult task such as standing on an unstable surface, the subject person might lean on a support even in a situation where the risk of fall is low.

As a result, a force touch (a contact load of 5N or greater) occurs, and not only a light touch effect cannot be achieved, but also erroneous posture control might be learned. In this case, even if sensory feedback is performed as disclosed in Non Patent Literature 1, the light touch state cannot be maintained.

The present invention has been made in view of the above aspects, and aims to provide a technology for maintaining a light touch state even in a case where the subject person performs a highly difficult task in which the subject person easily loses his/her posture.

### Solution to Problem

The technology disclosed herein provides a light touch maintaining device that includes:

a contact load evaluation unit that determines whether a subject person is in a light touch state, on the basis of a contact load between the subject person and a support; and

an exercise intervention control unit that controls exercise intervention on the subject person, to maintain a light touch state when the subject person is not in a light touch state.

### ADVANTAGEOUS EFFECTS OF INVENTION

The technology disclosed herein provides a technology for maintaining a light touch state even in a case where a highly difficult task in which the subject person easily loses his/her posture is performed.

### BRIEF DESCRIPTION OF DRAWINGS

FIG. 1 is a diagram illustrating the functional configuration of a light touch maintaining device.

FIG. 2 is a configuration diagram regarding implementation of a light touch maintaining system.

FIG. 3 is a detailed configuration diagram of a light touch maintaining device.

FIG. 4 is a flowchart of processing.

FIG. 5 is a diagram illustrating a target exercise intervention intensity determination model.

FIG. 6 is a diagram illustrating an example hardware configuration of a light touch maintaining device.

### DESCRIPTION OF EMBODIMENTS

The following is a description of an embodiment (the present embodiment) of the present invention, with reference to the drawings. The embodiment described below is merely an example, and embodiment to which the present invention is applied are not limited to the embodiment described below. In the description below, the target for which a light touch state is maintained is a person. The target for which a light touch state is maintained will be referred to as the “subject person”.

### Overview of the Embodiment

In the present embodiment, when the risk of fall is low, and a light touch state cannot be maintained, a light touch state is maintained not by a sensory feedback to the subject person but by exercise intervention on a fingertip of the subject person. Further, the risk of fall is taken into consideration in the exercise intervention design, so that exercise intervention is not forcibly performed when the risk of fall of the subject person is high.

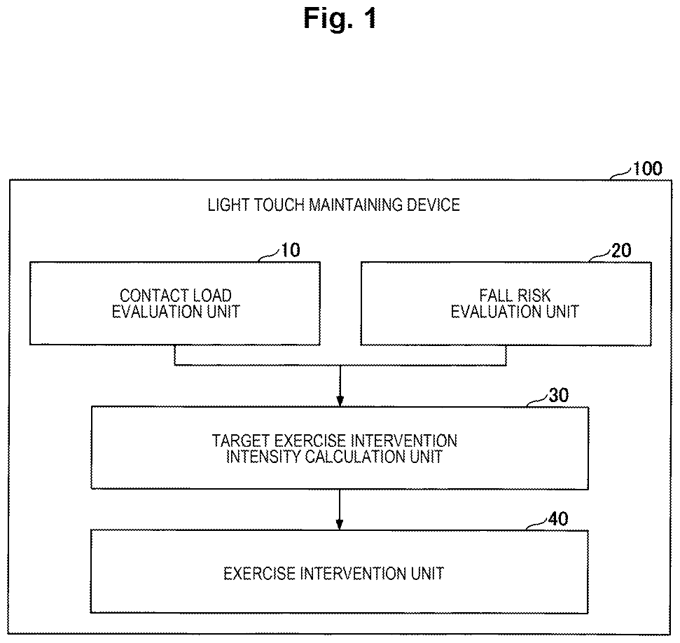

FIG. 1 illustrates an example functional configuration of a light touch maintaining device 100 according to the present embodiment. Note that the functional configuration illustrated in FIG. 1 corresponds to a functional configuration for performing processing in the PC described later.

As illustrated in FIG. 1, the light touch maintaining device 100 according to the present embodiment includes a contact load evaluation unit 10, a fall risk evaluation unit 20, a target exercise intervention intensity calculation unit 30, and an exercise intervention unit 40. An overview of each component is as described below. The target exercise intervention intensity calculation unit 30 and the exercise intervention unit 40 may be collectively referred to as an exercise intervention control unit.

The contact load evaluation unit 10 evaluates whether the contact state is a light touch state, on the basis of the contact load between the fingertip of the subject person and the support. In other words, it is determined whether the subject person is in a light touch state.

The fall risk evaluation unit 20 calculates a parameter to be the base for evaluating the risk of fall of the subject person, on the basis of the barycentric position of the subject person and the floor reaction force, and calculates a fall risk evaluation index.

The target exercise intervention intensity calculation unit 30 calculates a target exercise intervention intensity on the basis of a finger contact load and the fall risk evaluation index, using a model for determining the target exercise intervention intensity.

The exercise intervention unit 40 calculates an exercise intervention parameter corresponding to the target exercise intervention intensity, and issues an instruction to the device that performs exercise intervention on the subject person.

In the description below, a more specific example of the light touch maintaining device 100 is described as Example.

### Example

In this example, a light touch state of the subject person in a case where the subject person performs a right-leg stand task is to be maintained. In the mode of the light touch in this example, the subject person brings the right index finger into contact with a load measuring instrument (a surface pressure sheet, for example) fixed onto a table about the height of the waist of the subject person. An electrical stimulator is used for performing exercise intervention on the subject person, and electrical stimulation is presented to the muscle (common digital extensor muscle, for example) that extends the index finger of the subject person.

### Example Implementation

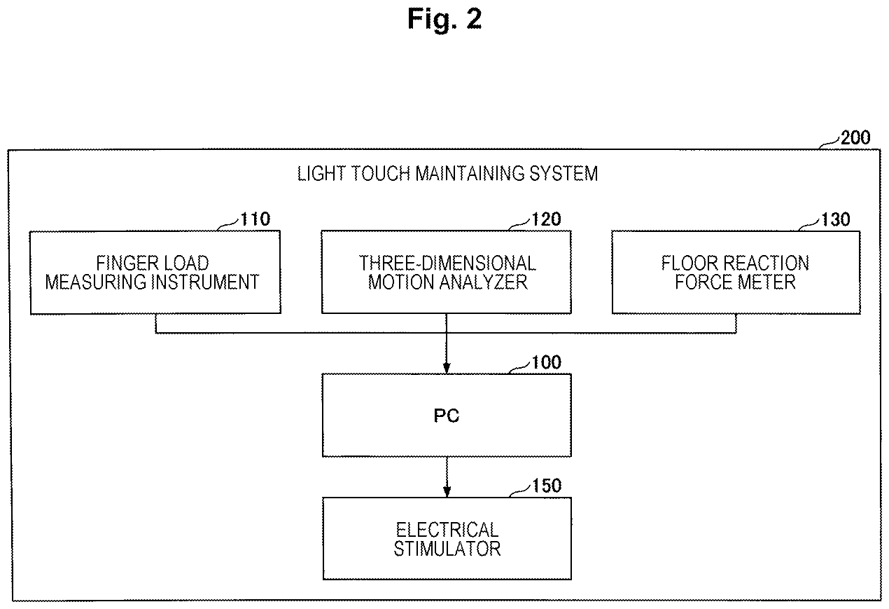

FIG. 2 illustrates example implementation regarding the light touch maintaining device 100 in this example. A PC 100 in FIG. 2 corresponds to the light touch maintaining device 100 illustrated in FIG. 1. Note that the entire configuration illustrated in FIG. 2 may be referred to as a light touch maintaining device or a light touch maintaining system. Here, the entire configuration illustrated in FIG. 2 is called a light touch maintaining system 200.

As illustrated in FIG. 2, the light touch maintaining system 200 in this example implementation includes a load measuring instrument 110, a three-dimensional motion analyzer 120, a floor reaction force meter 130, a personal computer (PC) 100, and an electrical stimulator 150. As illustrated in FIG. 2, the load measuring instrument 110, the three-dimensional motion analyzer 120, and floor reaction force meter 130 are connected to the PC 100.

The load measuring instrument 110 measures a contact load on a fingertip. The three-dimensional motion analyzer 120 and the floor reaction force meter 130 measure the gravity center of body (COM), the ground reaction force (GRF), and the center of ground pressure (COP) to be used in evaluating the risk of fall. Note that, from the measurement data obtained by the three-dimensional motion analyzer 120 and the floor reaction force meter 130, the PC 100 may calculate the gravity center of body (COM), the ground reaction force (GRF), and the center of ground pressure (COP).

The PC 100 evaluates the light touch state and the risk of fall of the subject person on the basis of information obtained from the load measuring instrument 110, the three-dimensional motion analyzer 120, and the floor reaction force meter 130, and determines the target exercise intervention intensity and the electrical stimulation parameter on the basis of the light touch state and the risk of fall. The electrical stimulator 150 presents electrical stimulation based on the electrical stimulation parameter corresponding to the target exercise intervention intensity determined by the PC 100, to the index-finger extensor muscle of the subject person.

### Specific Example Configuration

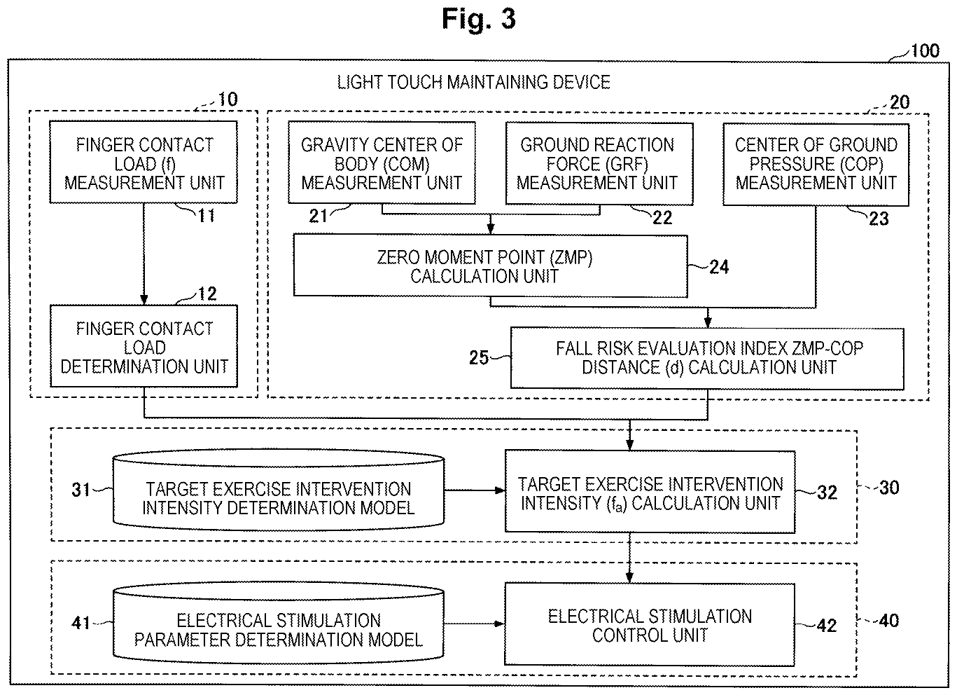

FIG. 3 illustrates a specific example configuration of the light touch maintaining device 100. FIG. 3 corresponds to a more detailed illustration of the respective components shown in the functional configuration illustrated in FIG. 1.

As illustrated in FIG. 3, the contact load evaluation unit 10 includes a finger contact load (f) measurement unit 11 and a finger contact load evaluation unit 12. Note that, in this example, the finger contact load (f) measurement unit 11 is a functional unit that receives a signal from the finger load measuring instrument 110 (such as a surface pressure sheet), and outputs a vertical load (f) to the finger contact load evaluation unit 12.

The fall risk evaluation unit 20 includes a gravity center of body (COM) measurement unit 21, a ground reaction force (GRF) measurement unit 22, a center of ground pressure (COP) measurement unit 23, a zero moment point (ZMP) calculation unit 24, and a fall risk evaluation index ZMP-COP distance (d) calculation unit 25.

Note that, in this example, the gravity center of body (COM) measurement unit 21 is a functional unit that calculates COM from data obtained by the three-dimensional motion analyzer 120, and the ground reaction force (GRF) measurement unit 22 and the center of ground pressure (COP) measurement unit 23 are functional units that calculate GRF and COP, respectively, from data obtained from the floor reaction force meter 130.

The target exercise intervention intensity calculation unit 30 includes a target exercise intervention intensity determination model 31 and a target exercise intervention intensity (fₐ) calculation unit 32. The exercise intervention unit 40 includes an electrical stimulation parameter determination model 41 and an electrical stimulation control unit 42.

### Processing Operation

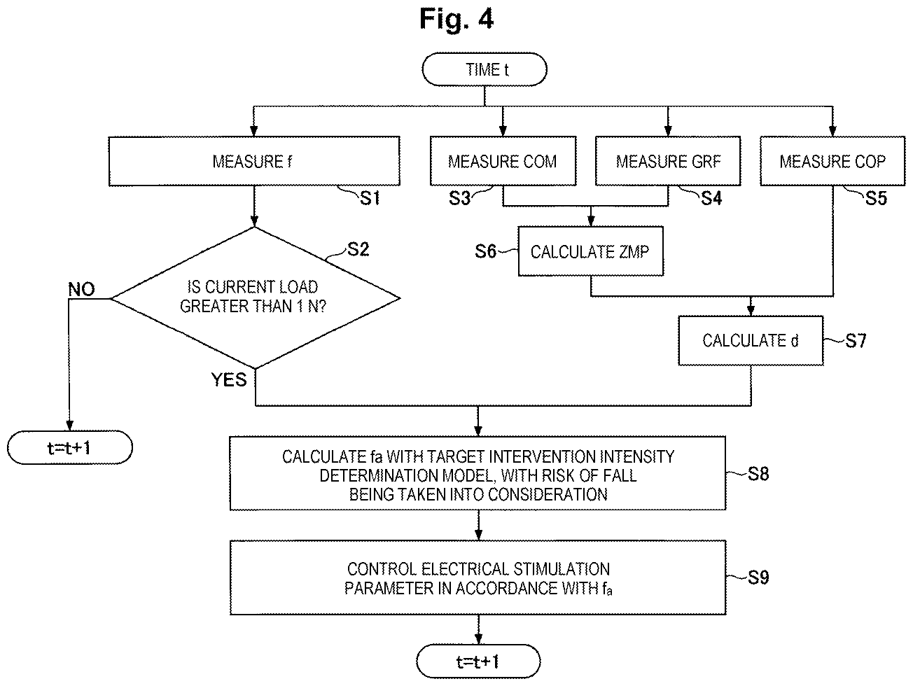

Next, an operation of the light touch maintaining device 100 illustrated in FIG. 3 is described with reference to a flowchart in FIG. 4. Note that, in FIG. 4 and the description below, f measurement, COM measurement, COP measurement, and the like are performed in order. However, this is for ease of explanation, and, in practice, these processes are performed in parallel.

### S1

In S1 (step 1), the finger contact load (f) measurement unit 11 measures the contact vertical load f(t) between the index finger of the subject person and the table at time t with a surface pressure sensor placed on the table at the height around the waist of the subject person, and outputs the measured contact vertical load f(t) to the finger contact load evaluation unit 12.

### S2

The finger contact load evaluation unit 12 compares the contact load f(t) between the fingertip of the subject person and the table as input from the finger contact load (f) measurement unit 11 with a threshold (1 N in this example), and determines whether the current contact load f(t) is larger than 1 N. Note that the threshold of 1 N is an example.

If f(t) is smaller than 1 N, t is updated to t+1, without any exercise intervention because of a light touch state. If f(t) is larger than 1 N, exercise intervention for a light touch state is required, and therefore, f(t) is output to the target exercise intervention intensity (fₐ) calculation unit 32. The process to be performed in the target exercise intervention intensity (fₐ) calculation unit 32 will be described later in S8.

### S3

In S3, the gravity center of body (COM) measurement unit 21 measures the position coordinates of a marker attached to the body of the subject person, using the three-dimensional motion analyzer 120. By doing so, the gravity center of body (COM) measurement unit 21 calculates the gravity center of body COM(t)=(COMₓ, COM_{y}, COM₂) at time t, and outputs the calculation result to the zero moment point (ZMP) calculation unit 24.

### S4

In S4, the ground reaction force (GRF) measurement unit 22 measures the ground reaction force GRF(t)=(Fₓ, F_{y}, F_{z}) of the subject person at time t with the floor reaction force meter 130, and outputs the calculation result to the zero moment point (ZMP) calculation unit 24.

### S5

In S5, the center of ground pressure (COP) measurement unit 23 measures the center of ground pressure COP (t)=(COPₓ, COP_{y}) of the subject person at time t with the floor reaction force meter 130, and outputs the measurement result to the fall risk evaluation index ZMP-COP distance (d) calculation unit 25.

### S6

In S6, the zero moment point (ZMP) calculation unit 24 calculates the zero moment point (ZMP) of the subject person, from COM(t) input from the gravity center of body (COM) measurement unit 21 and GRF(t) input from the ground reaction force (GRF) measurement unit 22.

The zero moment point is a virtual point on the floor surface, at which the sum of moments acting on the gravity center of body is zero. That is, when ZMP is equal to COP, a rotational moment that causes a fall does not act on the body center of gravity, which means that the posture is most stable. Here, the body when a person is in a stationary upright posture is regarded as a single inverted pendulum, and ZMP (t)=(ZMPₓ, ZMP_{y}) is calculated from the balance of moments around the gravity center of body, according to Expressions (1) and (2) shown below. The calculation result is output to the fall risk evaluation index ZMP-COP distance (d) calculation unit 25.

ZMPₓ(t)=COMₓ−(Fₓ/F_{z})COM₂ (1)

MP_{y}(t)=COM_{y}−(F_{y}/F_{z})COM_{z} (2)

### S7

In S7, the fall risk evaluation index ZMP-COP distance (d) calculation unit 25 calculates the fall risk evaluation index, from COP(t) input from the center of ground pressure (COP) measurement unit 23 and ZMP (t) input from the zero moment point (ZMP) calculation unit 24.

In this example, as the fall risk evaluation index, the distance d(t) between ZMP and COP is calculated according to Expressions (3) to (5) shown below. The larger d is, the more unstable the posture is likely to become, which means that the risk of fall is high. The calculated d(t) is output to the target exercise intervention intensity (fₐ) calculation unit 32.

dₓ(t)=ZMPₓ−COPₓ (3)

d_{y}(t)=ZMP_{y}−COP_{y} (4)

[Mathematical Expression 1]

d(t)=\sqrt{dₓ²+d_{y}²} (5)

### Target Exercise Intervention Intensity Determination Model

The target exercise intervention intensity determination model to be used in determining the target exercise intervention intensity fₐ(t) is now described.

The target exercise intervention intensity fₐ(t) is the target intensity of the exercise intervention for bringing the contact load on the fingertip of the subject person into a light touch state (1 N or smaller), with the risk of fall being taken into consideration.

The possible range of fₐ is f−1<fₐ<f, on the basis of 0<f−fₐ<1. In this example, the model shown below in which the target exercise intervention intensity fₐ varies with the fall risk evaluation index d is set as an example.

$$\begin{matrix}[ Mathematical Expression 2 ] & \\ \begin{matrix}{f}_{a} ( t ) = f ( 0 < d < {d}_{L} ) \\ {f}_{a} ( t ) = \frac{{d}_{L} - d}{{d}_{H} - {d}_{L}} + f ( {d}_{L} < d < {d}_{H} ) \\ {f}_{a} ( t ) = 0 ( {d}_{H} < d )\end{matrix} \} & ( 6 )\end{matrix}$$

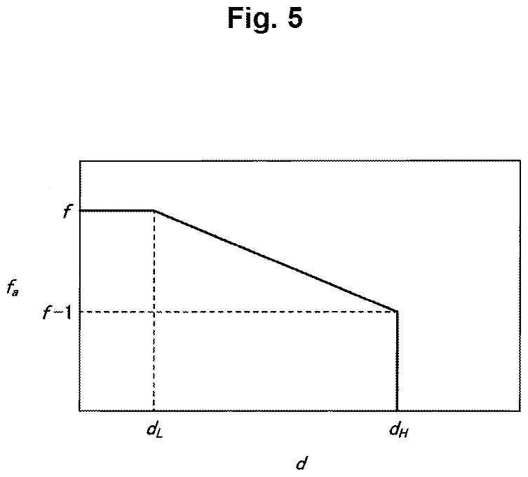

The model expressed by the above mathematical expression is illustrated in FIG. 5. As illustrated in FIG. 5, fₐ is determined depending on d. Note that, in a case where d is larger than d_{H} (where the risk of fall of the subject person has increased), exercise intervention is not performed.

Here, d_{L} and d_{H} are determined by conducting a preliminary experiment as follows.

d_{L}: the maximum value of d when standing still on both feet on a hard floor surface, with eyes open

d_{H}: the maximum value of d when standing on one leg on a soft floor surface, with eyes closed

Note that, in a case where the balance cannot be maintained halfway under the condition for determining d_{H}, d_{H} is a value that is 70% of d at that moment. Note that “70%” is an example.

The set target exercise intervention intensity determination model is stored into a storage device such as a memory.

### S8

In S8, to the target exercise intervention intensity (fₐ) calculation unit 32, the contact load f(t) (>1 N) between the finger and the table is input from the finger contact load evaluation unit 12, the fall risk evaluation index d(t) is input from the fall risk evaluation index ZMP-COP distance (d) calculation unit 25, and the target exercise intervention intensity determination model read from the storage device is input. Using these, the target exercise intervention intensity (fₐ) calculation unit 32 calculates the target exercise intervention intensity (fₐ) for the subject person.

The target exercise intervention intensity (fₐ) calculation unit 32 calculates the target exercise intervention intensity fₐ(t) by applying the current f and d to the target exercise intervention intensity determination model, and outputs fₐ(t) to the electrical stimulation control unit 42.

### Electrical Stimulation Parameter Determination Model

A determination model for determining the electrical stimulation parameter corresponding to the electrical stimulation for generating the target exercise intervention intensity at a fingertip is now described.

In an example, the pulse width is set to 200 us, the current is set to 10 mA, and the stimulation intensity is adjusted by changing the frequency between 0 and −200 Hz. A change in the load on the fingertip when the frequency is changed as a preliminary experiment, so that an electrical stimulation parameter determination model depending on the target exercise intervention intensity is created. The created electrical stimulation parameter determination model is stored into a storage device such as a memory.

### S9

In S9, the target exercise intervention intensity fₐ(t) is input from the target exercise intervention intensity (fₐ) calculation unit 32 to the electrical stimulation control unit 42, and the electrical stimulation parameter determination model read from the storage device is input thereto. The electrical stimulation control unit 42 determines the electrical stimulation parameter by applying fa to the model, and gives the determined electrical stimulation parameter to the electrical stimulator 150, to cause the electrical stimulator 150 to perform exercise intervention on the fingertip.

In this example, the contact portion in contact with the support of the subject person is one fingertip portion. However, a plurality of exercise intervention units may be provided, and both hands may be brought into contact with fixed points on parallel bars or the like, for example. Further, the position at which the load measuring instrument 110 is mounted may be changed to a fingertip, a palm, a wall, a safety bar, or the like. The target exercise intervention intensity determination model may be appropriately changed, with the fall risk evaluation index being the gravity center sway rate or the area. The device that performs exercise intervention on the subject person may also be replaced with a glove equipped with a vibrator or the like.

### Example Hardware Configuration

The light touch maintaining device 100 can be implemented by causing a computer to execute a program, for example. This computer may be a physical computer, or may be a virtual machine in a cloud.

Specifically, the light touch maintaining device 100 can be implemented by executing a program corresponding to the processing to be performed in the light touch maintaining device 100, using hardware resources such as a CPU and a memory included in the computer. The above program is recorded in a computer-readable recording medium (such as a portable memory) so that the program can be stored and distributed. Also, the program can be provided through a network such as the Internet or electronic mail.

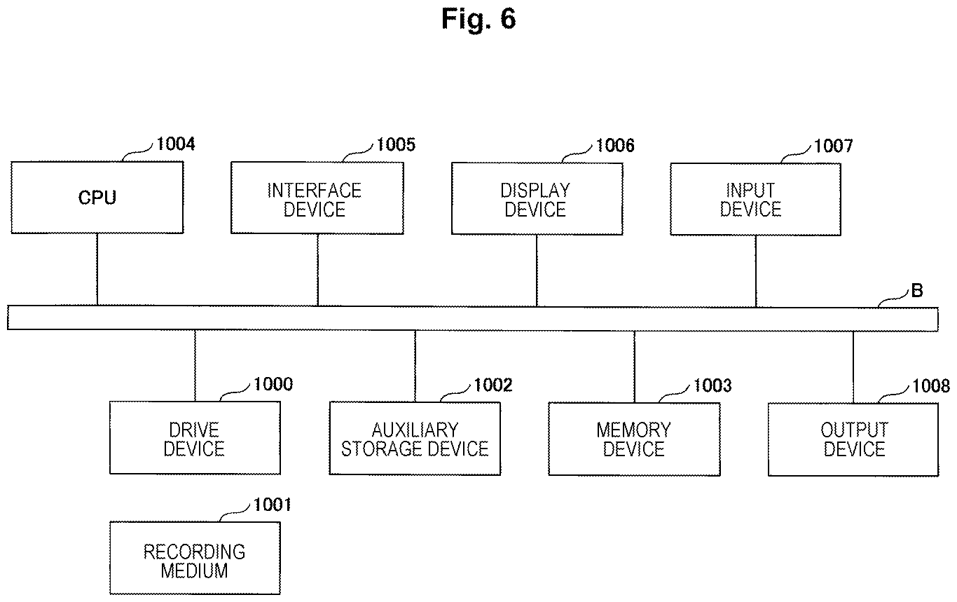

FIG. 6 is a diagram illustrating an example hardware configuration of the above computer. The computer in FIG. 6 includes a drive device 1000, an auxiliary storage device 1002, a memory device 1003, a CPU 1004, an interface device 1005, a display device 1006, an input device 1007, and an output device 1008, which are connected to one another by a bus BS.

The program for performing processing in the computer is provided through a recording medium 1001 such as a CD-ROM or a memory card, for example. When the recording medium 1001 that stores the program is set in the drive device 1000, the program is installed from the recording medium 1001 into the auxiliary storage device 1002 via the drive device 1000. However, the program is not necessarily installed through the recording medium 1001, but may be downloaded from another computer via a network. The auxiliary storage device 1002 stores the installed program, and also stores necessary files, data, and the like.

In a case where an instruction to start the program is issued, the memory device 1003 reads and stores the program from the auxiliary storage device 1002. The CPU 1004 implements the functions related to the light touch maintaining device 100, according to the program stored in the memory device 1003. The interface device 1005 is used as an interface for connection to the network, various measurement devices, an exercise intervention device, and the like. The display device 1006 displays a graphical user interface (GUI) or the like according to the program. The input device 1007 includes a keyboard and mouse, buttons, a touch panel, or the like, and is used to input various operation instructions. The output device 1008 outputs calculation results.

### Effects of the Embodiment

By the technology according to the present embodiment, it is possible to perform training while maintaining a light touch state even in a highly difficult task in which the posture is easily lost. Thus, it is possible to contribute to maximizing the posture control learning effect for the subject person, and improving the balance ability.

### Summary of the Embodiment

This specification discloses at least the light touch maintaining device, the light touch maintaining method, and the program according to the items described below.

### Item 1

A light touch maintaining device including:

a contact load evaluation unit that determines whether a subject person is in a light touch state, on the basis of a contact load between the subject person and a support; and

an exercise intervention control unit that controls exercise intervention on the subject person, to maintain a light touch state when the subject person is not in a light touch state.

### Item 2

The light touch maintaining device according to item 1, further including

a fall risk evaluation unit that evaluates a risk of fall of the subject,

wherein the exercise intervention control unit controls the exercise intervention on the subject person, on the basis of a result of evaluation performed by the fall risk evaluation unit.

### Item 3

The light touch maintaining device according to item 2, wherein

the fall risk evaluation unit calculates a distance between the zero moment point of the subject person and the center of ground pressure of the subject person, and

the exercise intervention control unit determines a target intensity of the exercise intervention on the subject person, on the basis of the distance.

### Item 4

The light touch maintaining device according to item 3, wherein

the exercise intervention control unit determines a parameter to be given to a device that performs the exercise intervention, on the basis of the target intensity.

### Item 5

A light touch maintaining method implemented by a light touch maintaining device,

the light touch maintaining method including:

a contact load evaluation step of determining whether a subject person is in a light touch state, on the basis of a contact load between the subject person and a support; and

an exercise intervention control step of controlling exercise intervention on the subject person, to maintain a light touch state when the subject person is not in a light touch state.

### Item 6

A program for causing a computer to function as each unit in the light touch maintaining device according to any one of items 1 to 4.

Although the present embodiment has been described above, the present invention is not limited to such a specific embodiment, and various modifications and changes can be made within the scope of the present invention disclosed in the claims.

### Reference Signs List

100 light touch maintaining device, PC

200 light touch maintaining system

110 load measuring instrument

120 three-dimensional motion analyzer

130 floor reaction force meter

150 electrical stimulator

10 contact load evaluation unit

20 fall risk evaluation unit

30 target exercise intervention intensity calculation unit

40 exercise intervention unit

11 finger contact load (f) measurement unit

12 finger contact load evaluation unit

21 gravity center of body (COM) measurement unit

22 ground reaction force (GRF) measurement unit

23 center of ground pressure (COP) measurement unit

24 zero moment point (ZMP) calculation unit

25 fall risk evaluation index ZMP-COP distance (d) calculation unit

31 target exercise intervention intensity determination model

32 target exercise intervention intensity (fa) calculation unit

41 electrical stimulation parameter determination model

42 electrical stimulation control unit

1000 drive device

1001 recording medium

1002 auxiliary storage device

1003 memory device

1004 CPU

1005 interface device

1006 display device

1007 input device

1. A light touch maintaining device comprising:
a processor; and
a memory that includes instructions, which when executed, cause the processor to execute:
determining whether a subject person is in a light touch state, on a basis of a contact load between the subject person and a support; and
controlling exercise intervention on the subject person, to maintain a light touch state when the subject person is not in a light touch state.

2. The light touch maintaining device according to claim 1, further comprising
evaluating a risk of fall of the subject,
wherein the controlling includes controlling the exercise intervention on the subject person, on a basis of a result of evaluation performed by the fall risk evaluation unit.

3. The light touch maintaining device according to claim 2, wherein
the evaluating includes calculating a distance between a zero moment point of the subject person and a center of ground pressure of the subject person, and
the controlling includes determining a target intensity of the exercise intervention on the subject person, on a basis of the distance.

4. The light touch maintaining device according to claim 3, wherein
the controlling includes determining a parameter to be given to a device that performs the exercise intervention, on a basis of the target intensity.

5. A light touch maintaining method implemented by a computer in a light touch maintaining device,
the light touch maintaining method comprising:
determining whether a subject person is in a light touch state, on a basis of a contact load between the subject person and a support; and
controlling exercise intervention on the subject person, to maintain a light touch state when the subject person is not in a light touch state.

6. A non-transitory computer-readable recording medium having computer-readable instructions stored thereon, which when executed. cause a computer including a memory and a processor to execute the determining and the controlling of the light touch maintaining device according to claim 1.
